(19) United States
(12) Patent Application Publication
(10) Pub. No.: US 2014/0081140 A1
(43) Pub. Date: Mar. 20, 2014

(54) ULTRASOUND IMAGING APPARATUS AND CONTROL METHOD FOR THE SAME

(71) Applicant: SAMSUNG ELECTRONICS CO., LTD., Suwon-si (KR)

(72) Inventors: Yun Tae KIM, Hwaseong-si (KR); Jung Ho KIM, Yongin-si (KR)

(73) Assignee: SAMSUNG ELECTRONICS CO., LTD., Suwon-si (KR)

(21) Appl. No.: 14/026,734

(22) Filed: Sep. 13, 2013

(30) Foreign Application Priority Data
Sep. 14, 2012 (KR) 10-2012-0102430

(51) Int. Cl. A61B 8/08

(52) U.S. Cl. CPC A61B 8/483
USPC 600/440

(57) ABSTRACT

Disclosed are an ultrasound imaging apparatus which includes a 3D display to display a 3D ultrasound image as well as a 2D display to display a 2D ultrasound image, thereby displaying both the anatomical shape of a diagnosis part and a high-resolution image, and a control method thereof. The ultrasound imaging apparatus includes an ultrasound data acquirer configured to acquire ultrasound data, a volume data generator configured to generate volume data based on the ultrasound data, a 3-Dimensional (3D) display image generator configured to generate a 3D ultrasound image based on the volume data, a cross-sectional image acquirer configured to acquire a cross-sectional image based on the volume data, a 3D display configured to display the 3D ultrasound image, and a 2D display configured to display the cross-sectional image.

### CROSS-REFERENCE TO RELATED APPLICATION

This application claims the benefit of Korean Patent Application No. 10-2012-0102430, filed on Sep. 14, 2012 in the Korean Intellectual Property Office, the disclosure of which is incorporated herein by reference in its entirety.

### BACKGROUND

1. Field

Exemplary embodiments of the present disclosure relate to an ultrasound imaging apparatus that outputs a 2-Dimensional (2D) ultrasound image and a 3-Dimensional (3D) ultrasound image of a subject, and a control method for the same.

2. Description of the Related Art

Ultrasound imaging apparatuses have non-invasive and non-destructive characteristics and are widely used in the field of medicine for acquisition of data regarding a subject. Recently developed ultrasound imaging apparatuses provide a 3D ultrasound image that provides spatial data and clinical data regarding a subject, such as an anatomical shape, etc., which are not provided by a 2D ultrasound image.

However, current ultrasound imaging apparatuses display a 3D ultrasound image on a 2D display unit, or display each cross-section of the 3D ultrasound image on a 2D display unit, which may make it difficult for an inspector to utilize substantial 3D effects of the 3D ultrasound image for diagnosis of diseases.

### SUMMARY

It is an aspect of the exemplary embodiments to provide an ultrasound imaging apparatus which includes a 3D display unit to display a 3D ultrasound image as well as a 2D display unit to display a 2D ultrasound image, thereby displaying both the anatomical shape of a diagnosis part and a high-resolution image, and a control method thereof.

Additional aspects of the exemplary embodiments will be set forth in part in the description which follows and, in part, will be obvious from the description, or may be learned by practice of the exemplary embodiments.

In accordance with an aspect of the exemplary embodiments, an ultrasound imaging apparatus includes an ultrasound data acquirer configured to acquire ultrasound data, a volume data generator configured to generate volume data from the ultrasound data, a 3-Dimensional (3D) display image generator configured to generate a 3D ultrasound image based on the volume data, a cross-sectional image acquirer configured to acquire a cross-sectional image based on the volume data, a 3D display configured to display the 3D ultrasound image, and a 2D display configured to display the cross-sectional image.

In accordance with another aspect of the exemplary embodiments, a control method for an ultrasound imaging apparatus includes acquiring ultrasound data regarding a subject, generating volume data regarding the subject based on the ultrasound data, generating a 2D cross-sectional image of the subject and a 3D ultrasound image of the subject based on the volume data, and displaying the 2D cross-sectional image of the subject on a 2D display and displaying the 3D ultrasound image of the subject on a 3D display.

### BRIEF DESCRIPTION OF THE DRAWINGS

These and/or other aspects of the exemplary embodiments will become apparent and more readily appreciated from the following description of the exemplary embodiments, taken in conjunction with the accompanying drawings of which:

FIG. 1 is a control block diagram illustrating an exemplary embodiment of an ultrasound imaging apparatus;

FIGS. 2A and 2B are perspective views illustrating an external appearance of an ultrasound imaging apparatus according to an exemplary embodiment;

FIG. 3 is a control block diagram illustrating an ultrasound data acquisition unit of the ultrasound imaging apparatus according to an exemplary embodiment;

FIG. 4 is a view illustrating a plurality of frame data constituting volume data according to an exemplary embodiment;

FIG. 5 is a control block diagram illustrating an exemplary embodiment of an ultrasound imaging apparatus;

FIG. 6 is a view illustrating a plurality of view-images generated by a view-image generator according to an exemplary embodiment;

FIG. 7 is a control block diagram illustrating another exemplary embodiment of an ultrasound imaging apparatus;

FIG. 8 is a view illustrating a configuration of a 3D display unit according to the exemplary embodiment of FIG. 7;

FIG. 9 is a control block diagram illustrating another exemplary embodiment of an ultrasound imaging apparatus;

FIG. 10 is a view illustrating display manipulators usable in an exemplary embodiment of an ultrasound imaging apparatus;

FIG. 11 is a control block diagram illustrating an exemplary embodiment of an ultrasound imaging apparatus that is configured to control the displaying of an image via motion recognition;

FIGS. 12A to 12C are views illustrating an exemplary embodiment of motion recognition;

FIG. 13 is a flowchart illustrating an exemplary embodiment of a control method for an ultrasound imaging apparatus; and

FIG. 14 is a flowchart illustrating a method of selecting a 2D cross-sectional image via motion recognition in the control method for the ultrasound imaging apparatus according to an exemplary embodiment.

### DETAILED DESCRIPTION

Reference will now be made in detail to an ultrasound imaging apparatus and a control method for the same according to the exemplary embodiments of the present disclosure, examples of which are illustrated in the accompanying drawings, wherein like reference numerals refer to like elements throughout.

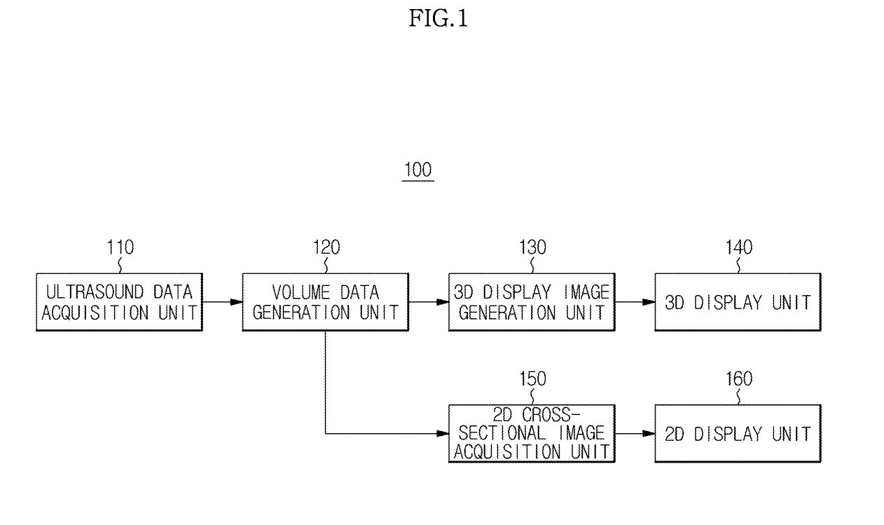

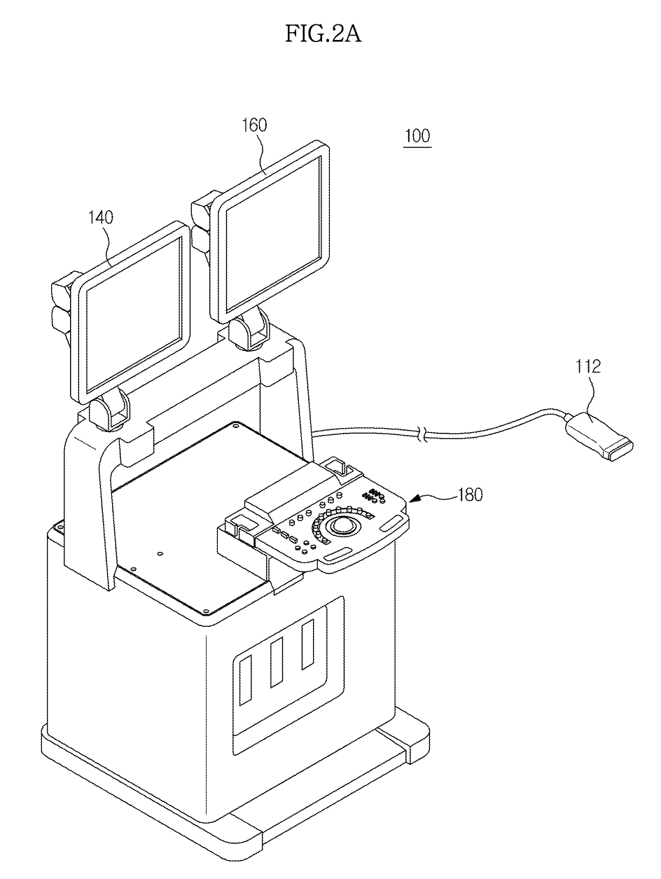

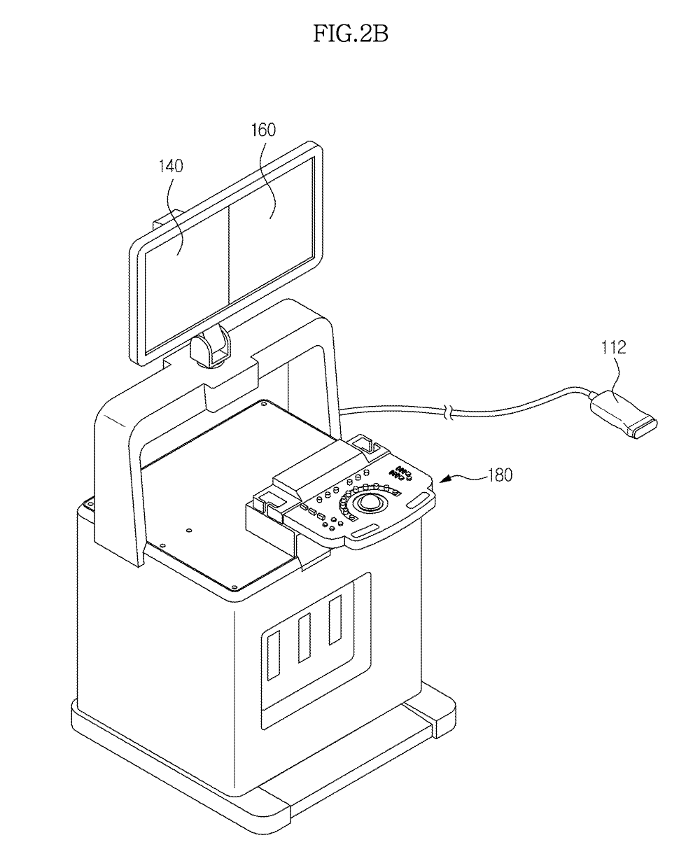

FIG. 1 is a control block diagram illustrating an exemplary embodiment of an ultrasound imaging apparatus, and FIGS. 2A and 2B are views illustrating an external appearance of the ultrasound imaging apparatus according to an exemplary embodiment.

Referring to FIG. 1, the ultrasound imaging apparatus 100 includes an ultrasound data acquisition unit 110 (e.g., ultrasound data acquirer) that acquires ultrasound data regarding a subject, a volume data generation unit 120 (e.g., volume data generator) that generates volume data regarding the subject, a 3D display image generation unit 130 (e.g., 3D display image generator) that generates an image to be output on a 3D display unit using the volume data regarding the subject, a 3D display unit 140, a cross-sectional image acquisition unit 150 (e.g., cross-sectional image acquirer) that acquires a 2D cross-sectional image from a 3D volume image, and a 2D display unit 160.

Referring to FIG. 2A, the 2D display unit 160 and the 3D display unit 140 may take the form of separate monitors or screens, and the monitors or the screens may be mounted respectively to a main body.

Alternatively, as illustrated in FIG. 2B, a single monitor or screen mounted to the main body may be divided into two areas, such that one area serves as the 2D display unit 160 and the other area serves as the 3D display unit 140.

The ultrasound imaging apparatus 100 displays a 3D ultrasound image of the subject on the 3D display unit 140 and a 2D ultrasound cross-sectional image regarding, for example, a diseased part of the subject on the 2D display unit 160, thereby simultaneously providing the anatomical shape of the subject and a high-resolution cross-sectional image for easy diagnosis of diseases.

The ultrasound imaging apparatus 100 includes an input unit 180 that receives an instruction from a user, such as, for example, an instruction based on a motion of the user. The user, e.g., an inspector such as a medical professional, may input an instruction for selection of a cross-sectional image or a variety of setting values with regard to generation of a 3D display image via the input unit.

Hereinafter, an operation of each constituent element of the ultrasound imaging apparatus according to exemplary embodiments will be described in detail.

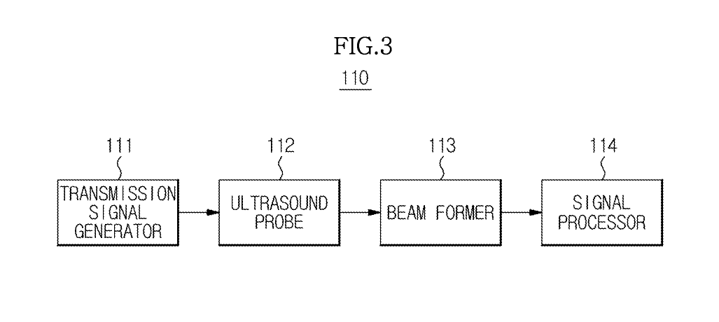

FIG. 3 is a control block diagram illustrating the ultrasound data acquisition unit included in the ultrasound imaging apparatus according to an exemplary embodiment.

Referring to FIG. 3, the ultrasound data acquisition unit 110 includes a transmission signal generator 111 that generates a transmission signal to be transmitted to the subject, an ultrasound probe 112 that transmits and receives ultrasonic signals to and from the subject, a beam-former 113 that generates focused reception signals upon receiving ultrasonic echo-signals received by the probe 112, and a signal processor 114 that generates ultrasound image data by processing the focused reception signals generated by the beam former 113.

The ultrasound probe 112 includes a plurality of transducer elements for changing between the use of ultrasonic signals and electric signals. To generate a 3D ultrasound image, a plurality of transducer elements may be arranged in a 2D array, or a plurality of transducer elements arranged in a 1D array may be swung in an elevation direction. Many different kinds of ultrasound probes may be implemented as the ultrasound probe 112 employed in the present exemplary embodiment so long as the ultrasound probe 112 may acquire a 3D ultrasound image.

Upon receiving the transmission signal from the transmission signal generator 111, the plurality of transducer elements changes the transmission signal into ultrasonic signals to transmit the ultrasonic signals to the subject. Then, the transducer elements generate reception signals upon receiving the ultrasonic echo-signals reflected from the subject. According to exemplary embodiments, the reception signals are analog signals.

More specifically, the ultrasound probe 112 appropriately delays an input time of pulses input to the respective transducer elements, thereby transmitting a focused ultrasonic beam to the subject along a scan line. Meanwhile, the ultrasonic echo-signals reflected from the subject are input to the respective transducer elements at different reception times, and the respective transducer elements output the input ultrasonic echo-signals.

To generate a 3D ultrasound image, signal generation in the transmission signal generator 111 and transmission and reception of the ultrasonic signals in the ultrasound probe 112 may be sequentially and iteratively performed, which enables sequential and iterative generation of reception signals.

The beam former 113 changes the analog reception signals transmitted from the ultrasound probe 112 into digital signals. Then, the beam former 113 receives and focuses the digital signals in consideration of positions and focusing points of the transducer elements, thereby generating focused reception signals. In addition, to generate a 3D ultrasound image, the beam former 113 sequentially and iteratively performs analog to digital conversion and reception-focusing according to the reception signals sequentially provided from the ultrasound probe 120, thereby generating a plurality of focused reception signals.

The signal processor 114, which may be implemented, for example, as a Digital Signal Processor (DSP), performs envelope detection processing to detect the strengths of the ultrasonic echo-signals based on the ultrasonic echo-signals focused by the beam former 113, thereby generating ultrasound image data. That is, the signal processor 114 generates ultrasound image data based on position data of a plurality of points present on each scan line and data acquired at the respective points. The ultrasound image data includes cross-sectional image data on a per scan line basis.

Referring again to FIG. 1, the volume data generation unit 120 generates volume data or a volume image of the subject via 3D reconstruction of multiple pieces of cross-sectional image data regarding the subject.

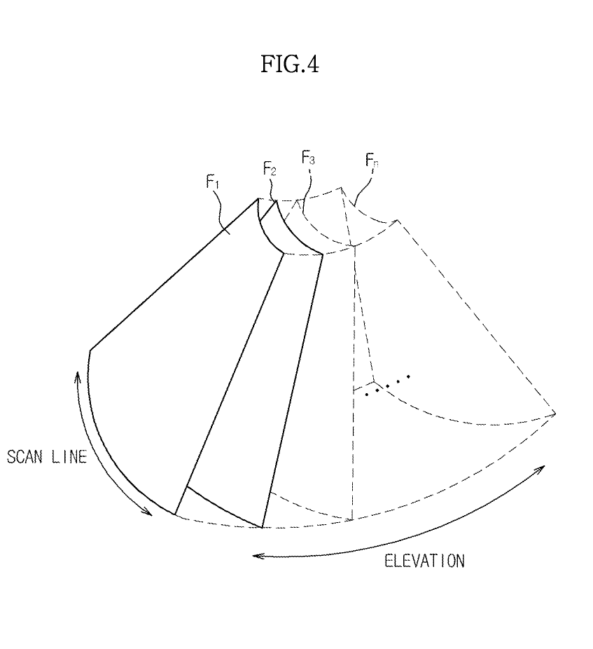

FIG. 4 illustrates a plurality of frame data constituting volume data. Referring to FIG. 4, each piece of cross-sectional image data generated by the signal processor 114 corresponds to frame data functioning as 2D ultrasound data. The volume data generation unit 120 may generate 3D volume data via data interpolation of a plurality of frame data F₁, F₂, F₃, . . . , Fₙ.

In consideration of the fact that volume data is generated by ultrasonic signals reflected from the subject that is present in a 3D space, the volume data according to exemplary embodiments is defined on a torus coordinate system. Accordingly, for rendering volume data via a display device having a Cartesian coordinate system such as a monitor, a scan conversion operation to convert coordinates of the volume data so as to conform to the Cartesian coordinate system may be performed. Accordingly, the volume data generation unit 120 may include a scan converter to convert coordinates of volume data.

The 3D display image generation unit 130 generates an image to be displayed on the 3D display unit 140 using a volume image of the subject. The 2D cross-sectional image acquisition unit 150 acquires a cross-sectional image of the subject from a volume image of the subject.

The 2D cross-sectional image acquisition unit 150 acquires a cross-sectional image to be displayed on the 2D display unit 160 from the volume image of the subject. The acquired cross-sectional image may be a cross-sectional image corresponding to the XY plane, the YZ plane, or the XZ plane, and may be an arbitrary cross-sectional image defined by the user. In addition, the cross-sectional image may be arbitrarily selected from the 2D cross-sectional image acquisition unit 150, or may be acquired in response to a cross-sectional image selection instruction input via the input unit 180 by the user. A detailed exemplary embodiment with regard to selection of the cross-sectional image will hereinafter be described.

The 3D display image generation unit 130 generates a 3D image conforming to an output format of the 3D display unit 140 such that the 3D image is displayed via the 3D display unit 140. Accordingly, the 3D image generated by the 3D display image generation unit 130 may be determined according to the output format of the 3D display unit 140.

The output format of the 3D display unit 140 may be various types, including, for example, a stereoscopic type, a volumetric type, a holographic type, an integral image type, or the like. The stereoscopic type is classified into a stereoscopic type using special glasses and a glasses-free auto-stereoscopic type.

Various exemplary embodiments with regard to generation of a 3D display image will hereinafter be described in detail. Ultrasound imaging apparatuses 200, 300, 400 and 500 of the exemplary embodiments that will be described hereinafter correspond to the ultrasound imaging apparatus 100 of the above-described exemplary embodiment, and the above description of the ultrasound imaging apparatus 100 may be applied to the ultrasound imaging apparatuses 200, 300, 400 and 500.

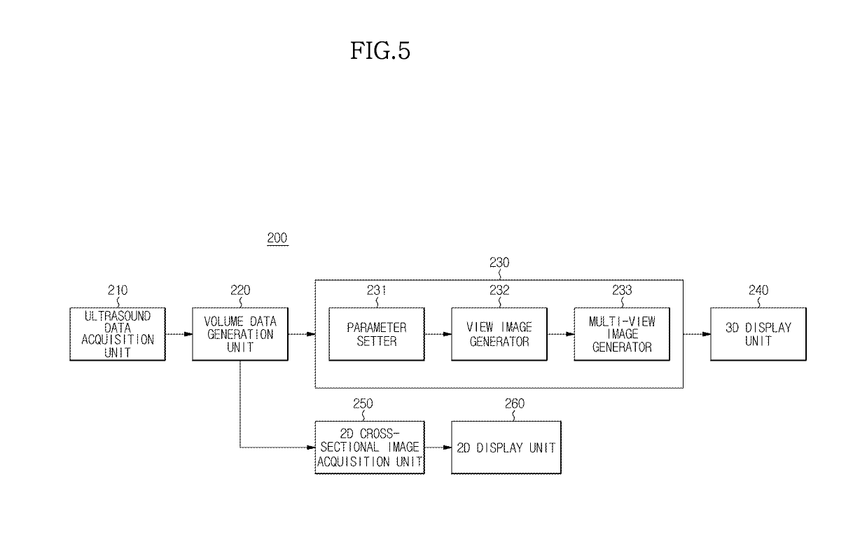

FIG. 5 is a control block diagram illustrating an exemplary embodiment of an ultrasound imaging apparatus.

An ultrasound data acquisition unit 210, a volume data generation unit 220, a 2D cross-sectional image acquisition unit 250, and a 2D display unit 260 may be substantially the same as the ultrasound data acquisition unit 110, the volume data generation unit 120, the 2D cross-sectional image acquisition unit 150 and the 2D display unit 160 described above with reference to FIGS. 1 to 3, and a description thereof is omitted herein.

A 3D display image generation unit 230 according to the present exemplary embodiment generates an autostereoscopic multi-view image.

Referring to FIG. 5, the 3D display image generation unit 230 includes a parameter setter 231 to set parameters regarding a view image, a view image generator 232 to generate a plurality of view images based on the set parameters, and a multi-view image generator 223 to generate a multi-view image using the plurality of view images.

The parameter setter 231 sets view parameters used to acquire a plurality of view images. In general, a multi-view image is generated by synthesizing images captured via a plurality of cameras. However, in the present exemplary embodiment, through the use of a program that acquires a virtual view image corresponding to a 3D volume image, view images, which are obtained as virtual cameras capture 3D volume data (3D volume images) generated by the volume data generation unit at different views, may be acquired. In this case, volume rendering may be used. According to exemplary embodiments, volume rendering may be performed by any one of various different types of rendering methods, such as Ray-Casting, Ray-Tracing, etc.

The view parameters used for generation of view images may include at least one of the number of views, view disparity, and a focal position. For example, the number of views may be determined according to characteristics of the 3D display unit 240, and view disparity and the focal position may be arbitrarily set by the parameter setter 231. Alternatively, the user may set setting values thereof via the input unit 180 illustrated in FIG. 2.

The view image generator 232 generates a plurality of view images having different views, which respectively correspond to the number of views, view disparity, and the focal position.

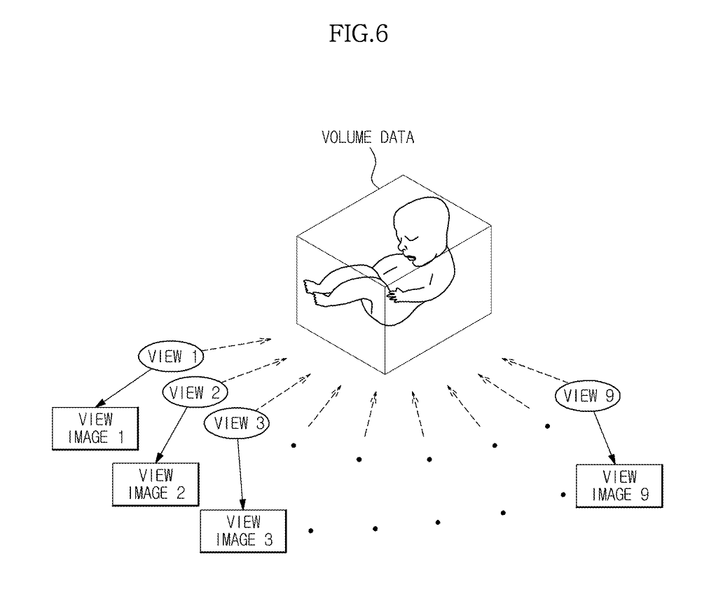

FIG. 6 is a view illustrating a plurality of view images generated by the view-image generator according to an exemplary embodiment.

Referring to FIG. 6, if the number of virtual cameras is set to 9, and positions of views, e.g., positions of the virtual cameras, are set to a constant-interval left-and-right arrangement on a horizontal axis under the assumption that the 3D display unit 240 has a characteristic of displaying 9 view images, the view-image generator 232 may generate 9 view images corresponding to the positions of the respective virtual cameras.

More specifically, the view image acquirer may acquire, using 3D volume data regarding the subject, a view-image 1 that may be acquired when capturing the subject by the camera located at the position of view 1 to a view-image 9 that may be acquired when capturing the subject by the camera located at the position of view 9.

The multi-view image generator 233 generates a multi-view image by synthesizing a plurality of view images acquired by the view image acquirer 232. According to exemplary embodiments, synthesizing a plurality of view images is referred to as weaving. Weaving generates a multi-view image by weaving a plurality of view images. When displaying the generated multi-view image, a viewer may perceive different 3D effects according to view positions where the viewer views an image. A detailed description of weaving is omitted.

When view disparity is set to a small value, a multi-view image having a small depth is generated. When view disparity is set to a large value, a multi-view image having a large depth is generated. The focal position may be set to a position behind the display unit 240, a position on the display unit 240, or a position in front of the display unit 240. As the focal position is displaced forward of the display unit 240, a multi-view image seems to protrude outward.

The generated multi-view image is displayed on the 3D display unit 240. When the multi-view image of the subject is displayed on the 3D display unit 240, the user may attain clinical data, such as the anatomical shape of the subject, at various views, which enables a diagnosis that is more accurate.

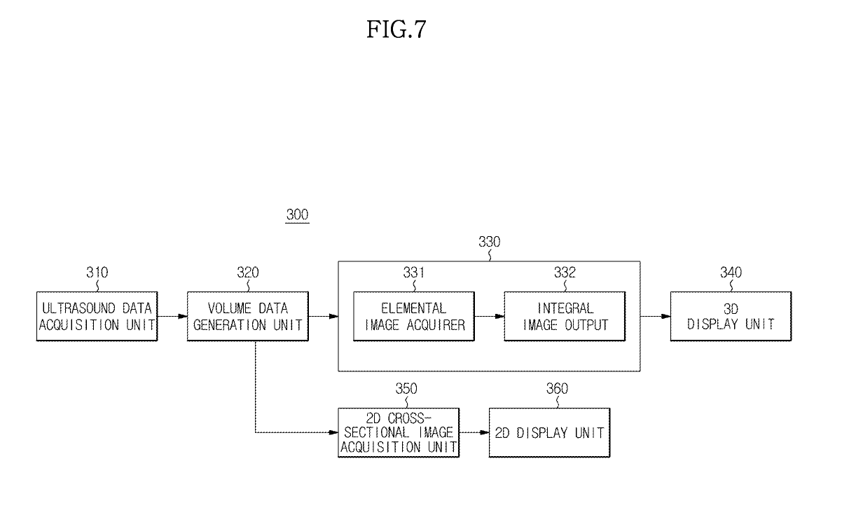

FIG. 7 is a control block diagram illustrating another exemplary embodiment of an ultrasound imaging apparatus.

An ultrasound data acquisition unit 310, a volume data generation unit 320, a 2D cross-sectional image acquisition unit 350, and a 2D display unit 360 may be substantially the same as the ultrasound data acquisition unit 110, the volume data generation unit 120, the 2D cross-sectional image acquisition unit 150 and the 2D display unit 160 described above with reference to FIGS. 1 to 3, and a description thereof is omitted herein.

The ultrasound imaging apparatus 300 according to the present exemplary embodiment generates an integral image of the subject, and displays the integral image on the 3D display unit 340. The integral image is acquired by storing 3D data of the subject in the form of elemental images using a lens array consisting of a plurality of elemental lenses, and integrating the elemental images into a 3D image via the lens array.

The integral image is an image having successive views in a left-and-right direction (horizontal direction) as well as in an up-and-down direction (vertical direction) within a view angle range, and may effectively transmit stereoscopic data regarding the subject to the user without requiring special glasses.

To generate the integral image, a pickup part to acquire elemental images of the subject and a display part to regenerate a 3D image from the acquired elemental images may be employed.

To this end, a 3D display image generation unit 330, as illustrated in FIG. 7, includes an elemental image acquirer 331 that acquires a plurality of elemental images of the subject, and an integral image output 332 that matches the acquired elemental images with the 3D display unit 340 to output an integral image. According to exemplary embodiments, the plurality of elemental images includes images having different horizontal parallaxes and vertical parallaxes.

Although the pickup part to acquire elemental images is generally constructed by a lens array and a plurality of cameras corresponding to the lens array, Computer Generated Integral Imaging (CGII) that calculates elemental images from 3D data regarding the subject using a computer program rather than actually capturing the elemental images has been proposed. Accordingly, the elemental image acquirer 331 may receive 3D volume data regarding the subject from the volume data generation unit 320, and may acquire elemental images of the subject under given conditions via imitation of the lens array based on a CGII program. The number of acquired elemental images and views of the elemental images may be determined according to the lens array of the 3D display unit 340.

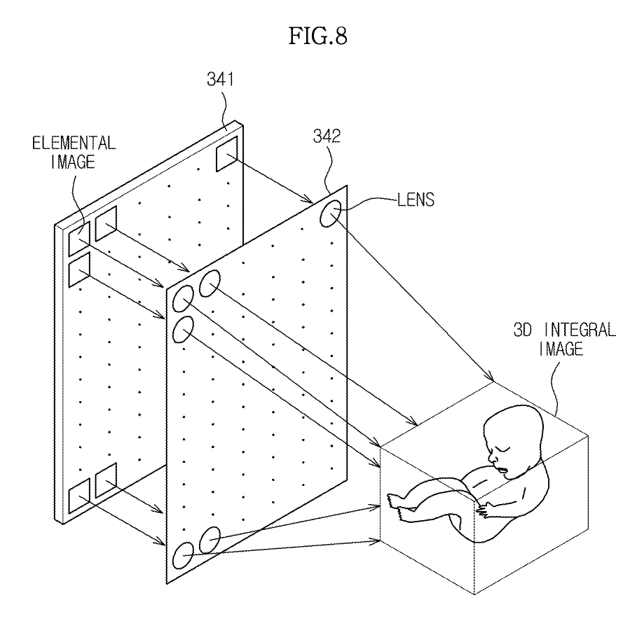

FIG. 8 is a view illustrating a configuration of the 3D display unit according to the exemplary embodiment of FIG. 7.

Referring to FIG. 8, the 3D display unit 340 may include a display device 341 that outputs elemental images, such as an LCD, a PDP, an LED, etc., and a lens array 342 that integrates the elemental images output via the display device 341 and generates a 3D image of the subject.

The integral image output 332 matches the elemental images acquired by the elemental image acquirer 331 with corresponding positions on the display device 341, thereby allowing the elemental images output via the display device 341 to be integrated by the lens array. As such, a 3D integral image of the subject may be generated.

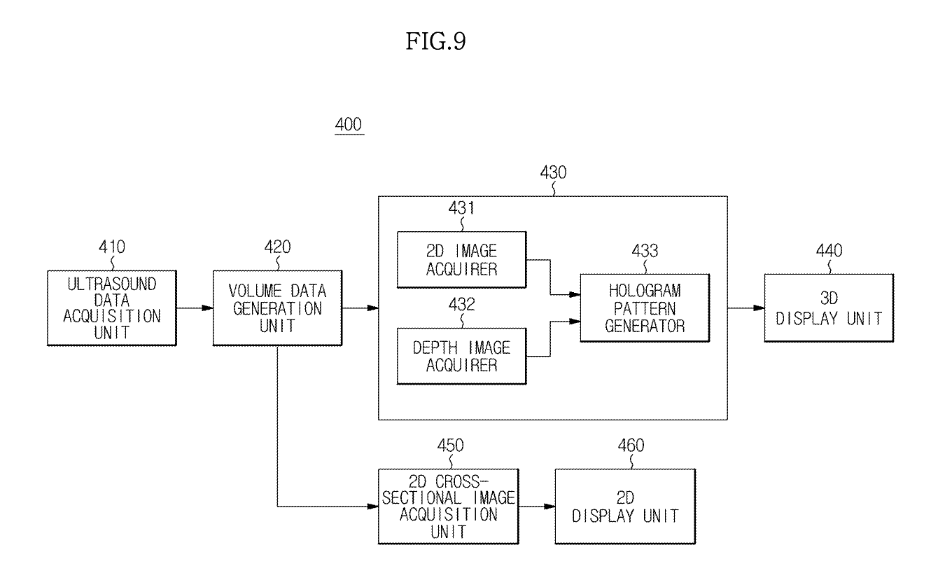

FIG. 9 is a control block diagram illustrating another exemplary embodiment of an ultrasound imaging apparatus.

An ultrasound data acquisition unit 410, a volume data generation unit 420, a 2D cross-sectional image acquisition unit 450, and a 2D display unit 460 may be substantially the same as the ultrasound data acquisition unit 110, the volume data generation unit 120, the 2D cross-sectional image acquisition unit 150 and the 2D display unit 160 described above with reference to FIGS. 1 to 4, and a description thereof is omitted herein.

The ultrasound imaging apparatus 400 of the present exemplary embodiment displays a 3D ultrasound image of the subject in a holographic manner, and a hologram generated in the holographic manner is referred to as a complete stereoscopic image. When recording a collision between object waves reflected from an object using laser light and laser light in another direction, an interference pattern depending on a phase difference of the object waves reflected from respective portions of the object is generated. An amplitude and a phase are recorded in the interference pattern. An image in which the shape of the object is recorded in the interference pattern is referred to as a hologram.

A 3D display image generation unit 430 may generate a hologram of the subject based on Computer Generated Holography (CGH). CGH is technology in which an interference pattern with respect to appropriate reference waves, e.g., a hologram, is calculated and generated using data of an object stored in a computer. CGH includes point-based CGH, convolution-based CGH, and Fourier-based CGH, for example. The 3D display image generation unit 430 may implement many different kinds of CGH to calculate and generate holograms.

Referring to FIG. 9, the 3D display image generation unit 430 includes a 2D image acquirer 431 to generate a 3D hologram of the subject, a depth-image acquirer 432, and a hologram pattern generator 433.

The 2D image acquirer 431 acquires a 2D image of the subject from a 3D volume image of the subject, and the depth-image acquirer 432 acquires a depth image of the subject from the 3D volume image of the subject. The 2D image of the subject may include color data regarding the subject.

The hologram pattern generator 433 generates a hologram pattern using a 2D image and a depth image regarding the subject. In an exemplary embodiment, the hologram pattern generator 433 may generate a single criterion elemental fringe pattern with respect to respective points of the subject that are equally spaced apart from a criterion point in a hologram plane. The criterion elemental fringe pattern may be pre-stored in a lookup table according to distances between the criterion points and the respective points of the subject. Alternatively, a criterion elemental fringe pattern on a per depth basis may be pre-stored.

The criterion elemental fringe pattern is shifted by a distance corresponding to the criterion elemental fringe pattern with respect to the respective points of the subject located in the same plane, so as to form a hologram pattern.

A 3D display unit 440 displays the generated hologram pattern to enable the user to view a 3D hologram of the subject.

The above-described exemplary embodiment of FIG. 9 is simply an example with regard to generation of a hologram, and other exemplary embodiments are not limited thereto. Various other methods for generation of a hologram of the subject may be applied to the present exemplary embodiment or other exemplary embodiments.

Operations of the ultrasound imaging apparatus for generation of the 3D ultrasound image of the subject and display of the 3D ultrasound image via the 3D display unit have been described above, and selection or control of an image to be displayed on each display unit will hereinafter be described.

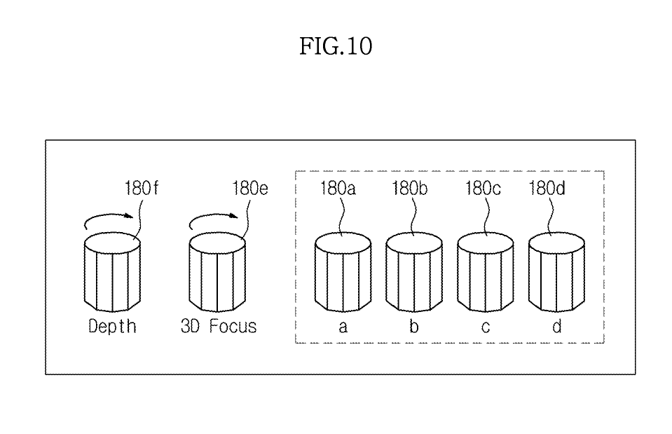

FIG. 10 is a view illustrating display manipulators usable in an exemplary embodiment of an ultrasound imaging apparatus.

As described above in FIGS. 2A and 2B, the ultrasound imaging apparatus 100 includes the input unit 180 that receives an instruction with regard to operations of the ultrasound imaging apparatus. As illustrated in FIG. 10, the input unit 180 may include a depth manipulator 180f that adjusts a depth of a 3D ultrasound image to be displayed on the 3D display unit 140, a focus manipulator 180e that adjusts a focus of the 3D ultrasound image, and cross-section manipulators 180a to 180d that select a 2D cross-sectional image. Each manipulator illustrated in FIG. 10 may be formed as buttons, and a setting value of the manipulator may be adjusted as the user rotates the manipulator by a predetermined angle, or may be directly input by the user.

The user may adjust a depth of a 3D ultrasound image via the depth manipulator 180f, and may adjust 3D effects of the 3D ultrasound image, e.g., a protrusion degree of the image on the basis of the display unit 140, via the focus manipulator 180e. As the 3D ultrasound image is controlled to project outward farther from the screen, 3D effects may be increased, but viewer eye fatigue may occur. In contrast, if the 3D ultrasound image is controlled to appear to be inserted into the display unit 140, 3D effects of the image are reduced, but the image is easy to diagnosis because extended viewing does not cause eye fatigue. Accordingly, a 3D ultrasound image of the subject may be controlled and displayed in an easily diagnosable form using each manipulator.

As described above, the ultrasound imaging apparatus 100 according to the exemplary embodiment may acquire a cross-sectional image from a 3D volume image of the subject and display the acquired image on the 2D display unit 160. In this case, although the cross-sectional image may be arbitrarily selected from the cross-sectional image acquisition unit 150, an instruction for selection of a cross-sectional image may also be input via the input unit 180 by the user. In an exemplary embodiment, the cross-section manipulators 180a to 180d illustrated in FIG. 10 may be used. The following Equation 1 may be used to represent a plane in space:

ax+by+cz+d=0 Equation 1

Here, the normal of a plane is represented by n=(a, b, c), and “d” represents a distance between the plane and a starting point. Accordingly, when the values a, b, c, and d are set respectively, a single plane is defined. The ultrasound imaging apparatus receives values of parameters a, b, c, and d that define a cross section from the user via the cross-section manipulators 180a to 180d, and acquires and displays a cross-sectional image corresponding to the values.

Alternatively, the 2D display unit 160 may take the form of a touchscreen, such that a portion of the touchscreen serves as an input unit. If the user, for example, drags a touch (e.g., user drags a finger contacting the touchscreen) from one point to another point, a cross-sectional image taken along the line connecting the two points to each other may be acquired.

If the user inputs an instruction to select a cross-sectional image, the 3D display unit 140 may display a 3D ultrasound image of the subject, or an image acquired via rendering of volume data on the 2D display unit 160. The user may refer to the displayed image for selection of the cross-sectional image.

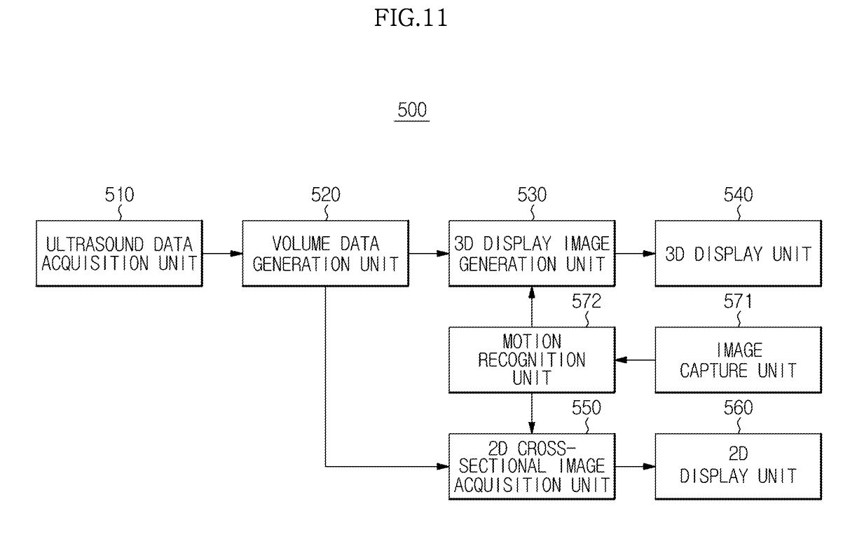

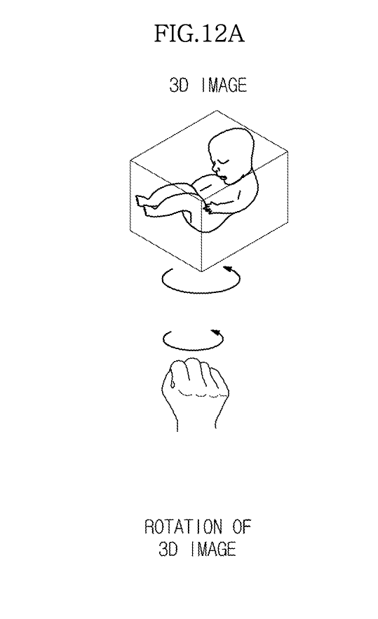

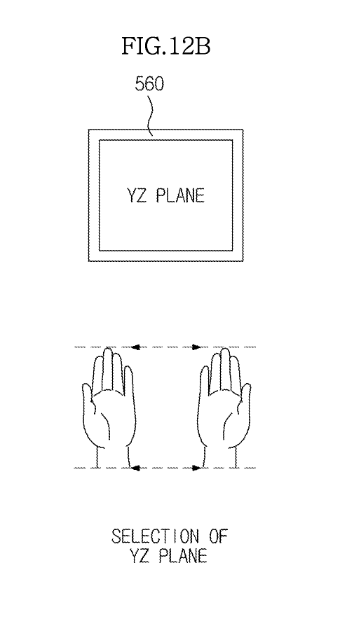

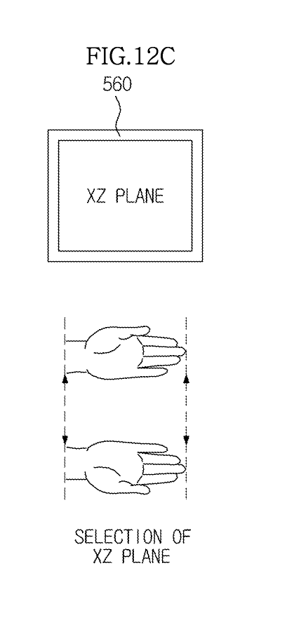

FIG. 11 is a control block diagram illustrating an exemplary embodiment of an ultrasound imaging apparatus that may control display of an image via motion recognition, and FIGS. 12A to 12C are views illustrating an exemplary embodiment of motion recognition.

Referring to FIG. 11, the ultrasound imaging apparatus 500 may include an image capture unit 571 (e.g., image capturer) that captures a user motion, and a motion recognition unit 572 that recognizes the user motion using the captured image.

The image capture unit 571 may be implemented as a camera, and may be mounted to a 2D display unit 560 or a 3D display unit 540. The image capture unit 571 captures an image of the user and transmits the image to the motion recognition unit 572. The motion recognition unit 572 recognizes a user motion by analyzing the captured image. The motion recognition unit 572 may be realized by any one of various motion recognition technologies. A detailed description of such motion recognition technologies is omitted herein.

In an exemplary embodiment, the motion recognition unit 572 may recognize the shape and motion of the user's hand. Instructions corresponding to the shape and motion of the user's hand may be preset. If the motion recognition unit 572 recognizes the preset shape and motion of the hand, a corresponding instruction may be transmitted to a 3D display image generation unit 530 or a 2D cross-sectional image acquisition unit 550.

For example, if the user rotates a clenched hand leftward or rightward as illustrated in FIG. 12A, a 3D image displayed on the 3D display unit 540 may be rotated according to the rotational direction of the hand.

Referring to FIG. 12B, if the user moves an open hand leftward or rightward in a state in which the user's fingers face upward, a cross-sectional image corresponding to the YZ plane may be extracted from a volume image of the subject, and may be displayed on the 2D display unit 560.

Referring to FIG. 12C, if the user moves an open hand upward or downward in a state in which the user's fingers face leftward or rightward, a cross-sectional image corresponding to the XZ plane may be extracted from a volume image of the subject, and may be displayed on the 2D display unit 560.

Motions illustrated in FIGS. 12A to 12C are given by way of example of motions that may be recognized by the motion recognition unit 572, and various other motions may be recognized to enable control of an image displayed on the display unit 560.

Hereinafter, an exemplary embodiment with regard to a control method of the ultrasound imaging apparatus will be described.

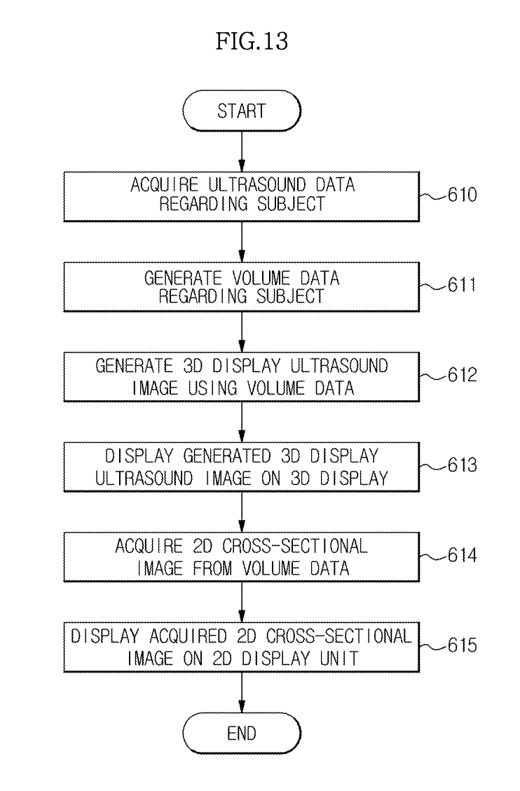

FIG. 13 is a flowchart illustrating an exemplary embodiment of a control method for the ultrasound imaging apparatus.

Referring to FIG. 13, first, ultrasound data regarding the subject is acquired at operation 610. To this end, a transmission signal is generated and transmitted to the ultrasound probe. The ultrasound probe changes the transmission signal into ultrasonic signals and transmits the ultrasonic signals to the subject, and then generates reception signals upon receiving ultrasonic echo-signals reflected from the subject. Then, signals input to the respective transducer elements of the ultrasound probe are focused to generate focused reception signals, and in turn, ultrasound data regarding the subject is acquired from the focused reception signals. The ultrasound data includes image data on a per scan line basis. In the present exemplary embodiment, a 3D ultrasound probe may be used to generate a 3D ultrasound image of the subject, and the 3D ultrasound probe may include a 2D array probe in which a plurality of transducer elements is arranged in a 2D form, and a 3D mechanical probe obtained by swing transducer elements of a 1D array, for example.

Next, volume data regarding the subject is generated from the acquired ultrasound data at operation 611. The volume data may be generated via 3D reconstruction of a plurality of pieces of cross-sectional image data regarding the subject.

A 3D display ultrasound image is generated using the volume data regarding the subject at operation 612. The 3D display ultrasound image is obtained by processing the volume data regarding the subject to conform to an output format of the 3D display unit.

In an exemplary embodiment, if the 3D display unit is configured to output a 3D multi-view image, a plurality of view images having different views is acquired from the volume data regarding the subject, and is synthesized to generate a multi-view image. In this case, weaving of the multi-view image may be implemented, and view disparity or the focal position as parameters for acquisition of view images may be set by the user.

In another exemplary embodiment, if the 3D display unit is configured to output an integral image, a plurality of elemental images having different horizontal parallaxes and vertical parallaxes is acquired from the volume data regarding the subject, and is matched with positions corresponding to a lens array of the 3D display unit.

In a further exemplary embodiment, if the 3D display unit is configured to output a hologram, a 2D image and a depth image are acquired from the volume data regarding the subject, and a hologram pattern is generated using the 2D image and the depth image.

Next, the generated 3D ultrasound image is displayed on the 3D display unit at operation 613. The 3D display unit may be of a stereoscopic type in which the viewer views a 3D image using special glasses, or of an auto-stereoscopic type in which the viewer views a 3D image without wearing special glasses. All of the above methods of generating the 3D display image, described by way of example with respect to operation 612, may be applied to the auto-stereoscopic type 3D display unit. In particular, if the 3D display unit is configured to directly output an image, the 3D display unit may include a display device, such as an LCD, LED, PDP, etc., and a lens array. As elemental images matched with the lens array are integrated by the lens array, a single 3D integral image is output.

A 2D cross-sectional image of the subject is displayed on the 2D display unit. To this end, a 2D cross-sectional image is acquired from the volume data regarding the subject at operation 614, and the acquired 2D cross-sectional image is displayed on the 2D display unit at operation 615. The acquired cross-sectional image may be a cross-sectional image corresponding to the XY plane, the YZ plane, or the XZ plane, or any other arbitrary images. Acquisition of the cross-sectional image may be performed by the ultrasound imaging apparatus, or may be performed in response to a selection instruction from the user. If a selection instruction is input by the user, a 3D ultrasound image of the subject may be displayed on the 3D display unit, or a volume image subjected to volume rendering may be displayed on the 2D display unit, so as to enable the user to select a 2D cross-sectional image based on the displayed image.

Although FIG. 13 illustrates the 2D ultrasound image as being displayed subsequent to display of the 3D ultrasound image, the exemplary embodiments are not limited as to the order of generation or display of the 3D ultrasound image and the 2D ultrasound image. Accordingly, any one of the two images may be initially generated or displayed, or the two images may be simultaneously generated or displayed.

When receiving the instruction for selection of the 2D cross-sectional image from the user, the instruction may be input via the input unit of the ultrasound imaging apparatus, or may be input via recognition of a user motion.

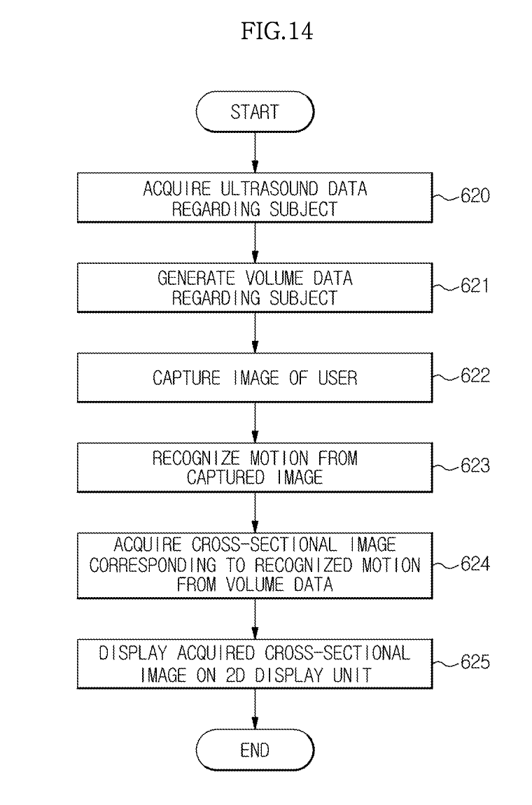

FIG. 14 is a flowchart illustrating a method of selecting a 2D cross-sectional image via motion recognition in the control method for the ultrasound imaging apparatus, according to an exemplary embodiment.

Referring to FIG. 14, ultrasound data regarding the subject is acquired at operation 620, and volume data regarding the subject is generated from the ultrasound data at operation 621. Acquisition of the ultrasound data and generation of the volume data may be performed in substantially the same fashion as operations 610 and 611, described above with respect to FIG. 13.

Next, an image of the user is captured using an image capture unit, such as a camera, etc., at operation 622. Motion recognition is performed based on the captured image at operation 623, and a cross-sectional image corresponding to the recognized motion is acquired from the volume data regarding the subject at operation 624. To this end, a particular motion and a cross-sectional image corresponding to the particular motion may be preset to correspond to each other. If a motion recognized from the captured image conforms to the preset particular motion, a corresponding cross-sectional image is acquired and displayed on the 2D display unit at operation 625.

As is apparent from the above description of exemplary embodiments, according to an ultrasound imaging apparatus and a control method for the same, both a 2D ultrasound image and a 3D ultrasound image are displayed, which may provide not only clinical data, such as the anatomical shape of a subject, but also a high-resolution image for diagnosis of diseases.

Although the exemplary embodiments of the present disclosure have been shown and described, it would be appreciated by those skilled in the art that changes may be made in these exemplary embodiments without departing from the principles and spirit of the present disclosure, the scope of which is defined in the claims and their equivalents.

What is claimed is:

1. An ultrasound imaging apparatus comprising:
an ultrasound data acquirer configured to acquire ultrasound data;
a volume data generator configured to generate volume data based on the ultrasound data;
a 3-Dimensional (3D) display image generator configured to generate a 3D ultrasound image based on the volume data;
a cross-sectional image acquirer configured to acquire a cross-sectional image based on the volume data;
a 3D display configured to display the 3D ultrasound image; and
a 2D display configured to display the cross-sectional image.

2. The apparatus according to claim 1, wherein the 3D display image generator comprises:
a view image generator configured to generate a plurality of view images having different respective views, based on the volume data; and
a multi-view image generator configured to generate a 3D multi-view image by synthesizing a plurality of the view images.

3. The apparatus according to claim 2, wherein the 3D display image generator further comprises a parameter setter configured to set a parameter to be used for generation of the view images, and
wherein the parameter includes at least one of view disparity and a focal position.

4. The apparatus according to claim 3, further comprising an input unit to receive a setting value of the parameter.

5. The apparatus according to claim 1, wherein the 3D display image generator:
an elemental image acquirer configured to acquire a plurality of elemental images based on the volume data; and
an integral image output configured to match the plurality of elemental images with respective corresponding positions on the 3D display so as to output an integral image.

6. The apparatus according to claim 5, wherein the 3D display comprises:
a display device to display the integral image output via the integral image output; and
a lens array including a plurality of lenses respectively corresponding to the plurality of elemental images.

7. The apparatus according to claim 1, wherein the 3D display image generator comprises:
a 2D image acquirer configured to acquire a 2D image of a subject based on the volume data;
a depth-image acquirer configured to acquire a depth-image of the subject based on the volume data; and
a hologram pattern generator configured to generate a hologram pattern using the 2D image of the subject and the depth-image of the subject.

8. The apparatus according to claim 1, further comprising an input configured to receive information indicating a selection of the cross-sectional image, and
wherein the cross-sectional image acquirer acquires the selected cross-sectional image from the volume data.

9. The apparatus according to claim 8, wherein the input is further configured to receive information to set at least one of a depth and focal position of the 3D ultrasound image to be displayed on the 3D display.

10. The apparatus according to claim 1, further comprising:
an image capturer configured to capture an image of a user; and
a motion recognition unit to recognize a motion from the captured image of the user.

11. The apparatus according to claim 10, wherein the motion recognition unit determines whether or not the recognized motion conforms to a preset motion, and transmits an instruction corresponding to the preset motion to the 3D display image generator or the 2D cross-sectional image acquirer if the recognized motion conforms to the preset motion.

12. A control method for an ultrasound imaging apparatus, the method comprising:
acquiring ultrasound data regarding a subject;
generating volume data regarding the subject based on the ultrasound data;
generating a 2D cross-sectional image of the subject and a 3D ultrasound image of the subject based on the volume data; and
displaying the 2D cross-sectional image of the subject on a 2D display and displaying the 3D ultrasound image of the subject on a 3D display.

13. The method according to claim 12, wherein the generating of the 3D ultrasound image comprises:
acquiring a plurality of view images having different respective views based on the volume data; and
generating a multi-view image by synthesizing the plurality of view images.

14. The method according to claim 13, further comprising receiving a parameter to set at least one of view disparity and a focal position with respect to each of the plurality of view images.

15. The method according to claim 12, wherein the generating of the 3D ultrasound image comprises:
acquiring a plurality of elemental images having different respective horizontal parallaxes and different respective vertical parallaxes, based on the volume data; and
matching the plurality of elemental images with respective corresponding positions on the 3D display.

16. The method according to claim 12, wherein the generating of the 3D ultrasound image comprises:
acquiring a 2D image and a depth-image based on the volume data; and
generating a hologram pattern using the 2D image and the depth-image.

17. The method according to claim 12, wherein the generating of the 2D cross-sectional image comprises receiving information indicating a selection of the 2D cross-sectional image from a user.

18. The method according to claim 12, wherein the generating of the 2D cross-sectional image comprises:
capturing an image of a user;
recognizing a motion from the image of the user; and
generating a cross-sectional image corresponding to the recognized motion.

19. The apparatus according to claim 1, further comprising:
a manipulator configured to receive a user's instruction to adjust a depth of the 3D ultrasound image or a focus of the 3D ultrasound image, or select a 2D cross-sectional image of the 3D ultrasound image.
